(19) United States
(12) Patent Application Publication
(10) Pub. No.: US 2008/0100208 A1
(43) Pub. Date: May 1, 2008

(54) ORGANIC LIGHT-EMITTING COMPOUND, ORGANIC LIGHT-EMITTING DEVICE INCLUDING THE COMPOUND, AND METHOD OF MANUFACTURING THE ORGANIC LIGHT-EMITTING DEVICE

(75) Inventors: Dong-woo SHIN, Yongin-si (KR); Woon-jung PAEK, Yongin-si (KR); Yi-yeol LYU, Yongin-si (KR); Myeong-suk KIM, Yongin-si (KR); Eun-sil HAN, Yongin-si (KR); Shinichiro TAMURA, Yongin-si (KR); Byoung-ki CHOI, Yongin-si (KR); O-hyun KWON, Yongin-si (KR); Sang-hoon PARK, Yongin-si (KR); Young-mok SON, Yongin-si (KR); Jung-bae SONG, Yongin-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/833,748

(22) Filed: Aug. 3, 2007

(30) Foreign Application Priority Data
Nov. 1, 2006 (KR) 10-2006-0107486

(51) Int. Cl. H01J 63/04; C07C 13/48; C07C 211/00; C07D 401/00; C07F 7/00; C07F 7/08; C09B 5/00

(52) U.S. Cl. 313/504; 546/167; 548/416; 556/406; 556/95; 564/433; 585/26

(57) ABSTRACT

Provided are a compound represented by Formula 1 below and an organic light-emitting device including the same:

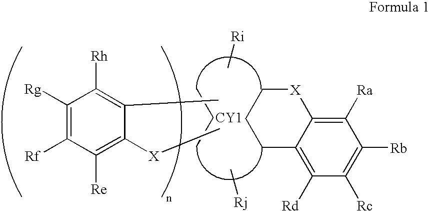

wherein X is a C, Si, or Ge atom disubstituted with H or C₁₋₆₀ organic groups, Rₐ-Rⱼ are C₁₋₆₀ organic groups, CY1 is a substituted or unsubstituted C₅-C₆₀ aromatic ring or a substituted or unsubstituted C₂-C₆₀ heteroaromatic ring, and n is 0 or 1. The use of the compound provides an organic light-emitting device having a low operating voltage and good efficiency and brightness.

This application claims priority to Korean Patent Application No. 10-2006-0107486, filed on Nov. 01, 2006, and all the benefits accruing therefrom under 35 U.S.C. § 119, the content of which is incorporated herein by reference in its entirety.

### BACKGROUND OF THE INVENTION

1. Field of the Invention

The present invention relates to an organic light-emitting compound and an organic light-emitting device including the same. More particularly, the present invention relates to an organic light-emitting compound that is excellent in electrical properties, thermal stability, and photochemical stability, and when applied to an organic light-emitting device, can offer excellent operating voltage and color purity characteristics, and an organic light-emitting device employing an organic layer including the compound.

2. Description of the Related Art

Light-emitting devices are self-emitting devices and have advantages such as a wide viewing angle, good contrast, and a rapid response time. Light-emitting devices are classified into inorganic light-emitting devices using a light-emitting layer formed of an inorganic compound and Organic Light-Emitting Devices (“OLEDs”) using a light-emitting layer formed of an organic compound. OLEDs show better brightness, operating voltage, and response speed characteristics and can create polychromatic light, in contrast to inorganic light-emitting devices, and thus, extensive research into OLEDs has been conducted.

Generally, OLEDs have a stacked structure which includes in sequence an anode, an organic light-emitting layer, and a cathode. OLEDs may also have varied structures such as, in sequence, an anode/hole injection layer/hole transport layer/emitting layer/electron transport layer/electron injection layer/cathode structure or an anode/hole injection layer/hole transport layer/emitting layer/hole blocking layer/electron transport layer/electron injection layer/cathode structure.

Materials used for OLEDs can further be categorized as vacuum-depositable materials or solution-coatable materials provided according to an organic layer formation process. Vacuum-depositable materials must have a vapor pressure of greater than or equal to 10⁻⁶ torr at a temperature of 500° C. or less, and are low molecular weight materials having a molecular weight of 1,200 g/mol or less. Solution-coatable materials must have sufficient solubility to form solutions, and can include primarily an aromatic or heterocyclic ring.

When manufacturing OLEDs using a vacuum deposition process, manufacturing costs may increase due to use of a vacuum system, and it may be difficult to manufacture high-resolution pixels for natural color displays due to a shadow mask. On the other hand, OLEDs can be manufactured using a solution coating process, such as for example, inkjet printing, screen printing, or spin coating, the manufacturing process is simple, manufacturing costs are low, and a relatively high resolution can be achieved compared to the resolution obtainable using a shadow mask.

However, when using solution-coatable materials, the performance (such as, thermal stability and color purity) of the light-emitting molecules, specifically blue light-emitting molecules, is reduced when compared to corresponding vacuum-depositable materials. Even though the light-emitting molecules of the solution-coatable materials have good performance, there problems which can arise in that the materials, when formed into an organic layer, gradually crystallize and grow into a size that is comparable to the visible light wavelength range so that, the grown crystals can scatter visible light. This can in turn cause a turbidity phenomenon so that pin holes, and like defects may form in the organic layer. Such defects can, thereby causing device performance degradation.

Japanese Patent Laid-Open Publication No. 1999-003782 discloses a two naphthyl-substituted anthracene compound that can be used in an emitting layer or a hole injection layer. However, the anthracene compound has poor solubility in solvents, and therefore when used, OLEDs employing the anthracene compound can have unsatisfactory characteristics.

Thus, it is desirable to develop a compound for use in OLEDs that can form a good organic layer irrespective of the organic layer formation process used.

Therefore, there remains a need to develop OLEDs with improved operating voltage, brightness, efficiency, and color purity characteristics based on blue light-emitting compounds which have good thermal stability and can form good organic layers.

### BRIEF SUMMARY OF THE INVENTION

In an embodiment, an organic light-emitting compound with good thermal stability is provided.

In another embodiment, an organic light-emitting device with improved operating voltage, efficiency, and brightness characteristics is provided.

Also, in another embodiment, a method of manufacturing the organic light-emitting device is provided.

In an embodiment, an organic light-emitting compound is represented by Formula 1, below:

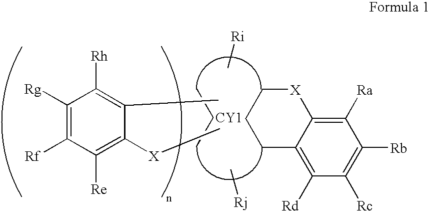

wherein X is a C, Si, or Ge atom disubstituted with H or C₁₋₆₀ organic groups, Rₐ-Rⱼ are C₁₋₆₀ organic groups, CY1 is a substituted or unsubstituted C₅-C₆₀ aromatic ring or a substituted or unsubstituted C₂-C₆₀ heteroaromatic ring, and n is 0 or 1.

Specifically, in an embodiment, an organic light-emitting compound is represented by Formula 2a below:

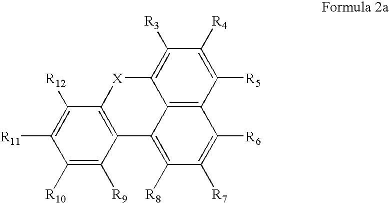

wherein X is C(R₁)(R₂), Si(R₁₃)(R₁₄), or Ge(R₁₃)(R₁₄) where R₁ and R₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, R₁ and R₂ may be connected to form a substituted or unsubstituted C₅-C₆₀ aromatic ring or a substituted or uhsubstituted C₅-C₆₀ aliphatic ring, R₁₃ and R₁₄ are each independently a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, and R₁₃ and R₁₄ may be connected to form a substituted or unsubstituted C₅-C₆₀ aromatic ring or a substituted or unsubstituted C₅-C₆₀ aliphatic ring; and

R₃, R₄, R₅, R₆, R₇, R₈, R₉, R₁₀, R₁₁, and R₁₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₆₀ arylamino group, or a substituted or unsubstituted C₁-C₆₀ alkylamino group, and two or more selected from R₃, R₄, R₅, R₆, R₇, R₈, R₉, R₁₀, R₁₁, and R₁₂ may be connected to form a fused substituted or unsubstituted C₆-C₆₀ aromatic ring or a fused substituted or unsubstituted C₆-C₆₀ heteroaromatic ring.

In another embodiment, an organic light-emitting device includes: a first electrode; a second electrode; and at least one organic layer interposed between the first electrode and the second electrode, the organic layer including the above-described organic light-emitting compound.

In another embodiment, a method of manufacturing an organic light-emitting device includes: forming a first electrode; forming on the first electrode an organic layer including an organic light-emitting compound according to an embodiment of the present invention; and forming a second electrode on the organic layer.

### BRIEF DESCRIPTION OF THE DRAWINGS

The above and other features and advantages of the present invention will become more apparent by describing in detail exemplary embodiments thereof with reference to the attached drawings in which:

FIGS. 1A through 1C are schematic sectional views illustrating exemplary organic light-emitting devices according to an embodiment;

FIG. 2 illustrates the UV and photoluminescence (“PL”) spectrum in solution of a compound 5 according to an exemplary embodiment; and

FIG. 3 is a graph illustrating voltage-efficiency characteristics of an organic light-emitting device sample 1 manufactured using a compound according to an exemplary embodiment.

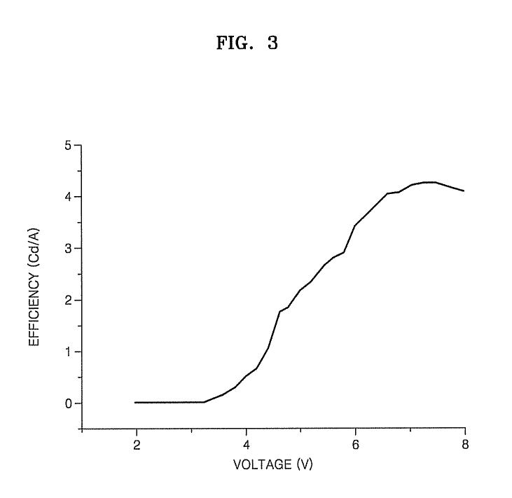

### DETAILED DESCRIPTION OF THE INVENTION

The present invention will now be described more fully with reference to the accompanying drawings, in which exemplary embodiments of the invention are shown.

It will be understood that when an element is referred to as being “on” another element, it can be directly on the other element or intervening elements may be present therebetween. In contrast, when an element is referred to as being “disposed on”, “interposed between”, or “formed on” another element, the elements are understood to be in at least partial contact with each other, unless otherwise specified.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As disclosed herein, an organic light emitting compound comprises a substituted or unsubstituted C₆-C₂₀₀ polycyclic aromatic compound having at least two aromatic groups Ar connected to each other by both an Ar—Ar single bond and by a bond connecting each Ar to a common, intervening disubstituted C, Si, or Ge atom.

In an embodiment, an organic light emitting compound is represented by Formula 1, below:

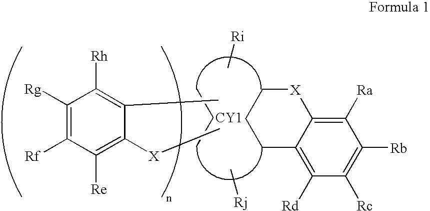

wherein X is a C, Si, or Ge atom disubstituted with H or C₁₋₆₀ organic groups, Rₐ-Rⱼ are C₁₋₆₀ organic groups, CY1 is a substituted or unsubstituted C₅-C₆₀ aromatic ring or a substituted or unsubstituted C₂-C₆₀ heteroaromatic ring, and n is 0 or 1. It will be understood that, for Formula 1 where n is 1, no particular connectivity of the floating bonds is implied that would lead to a specific substitution pattern and symmetry group (if any) for the resulting structure, unless otherwise specified.

In a specific embodiment, an organic light-emitting compound is represented by Formula 2a below:

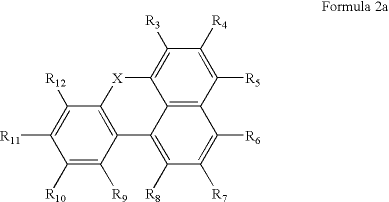

wherein X is C(R₁)(R₂), Si(R₁₃)(R₁₄), or Ge(R₁₃)(R₁₄) where R₁ and R₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, R₁ and R₂ may be connected to form a substituted or unsubstituted C₅-C₆₀ aromatic ring or a substituted or unsubstituted C₅-C₆₀ aliphatic ring, R₁₃ and R₁₄ are each independently a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, and R₁₃ and R₁₄ may be connected to form a substituted or unsubstituted C₅-C₆₀ aromatic ring or a substituted or unsubstituted C₅-C₆₀ aliphatic ring; and

R₃, R₄, R₅, R₆, R₇, R₈, R₉, R₁₀, R₁₁, and R₁₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₆₀ arylamino group, or a substituted or unsubstituted C₁-C₆₀ alkylamino group, and two or more selected from R₃, R₄, R₅, R₆, R₇, R₈, R₉, R₁₀, R₁₁, and R₁₂ may be connected to form a fused substituted or unsubstituted C₆-C₆₀ aromatic ring or a fused substituted or unsubstituted C₆-C₆₀ heteroaromatic ring.

In Formula 2a above, R₁ and R₂ serve to increase the solubility and amorphous characteristics of the organic light-emitting compound of Formula 2a above to thereby enhance film proccesability. The organic light-emitting compound of Formula 2a above is suitable as a material constituting an organic layer interposed between a first electrode and a second electrode of an organic light-emitting device. The organic light-emitting compound of Formula 2a above is suitable to be used in an organic layer of an organic light-emitting device, in particular, an emitting layer, a hole injection layer, a hole blocking layer, an electron transport layer, or a hole transport layer. The organic light-emitting compound of Formula 2a above may also be used as a host material or a dopant material.

In another specific embodiment, an organic light-emitting compound may be represented by Formula 2b or 2c, below:

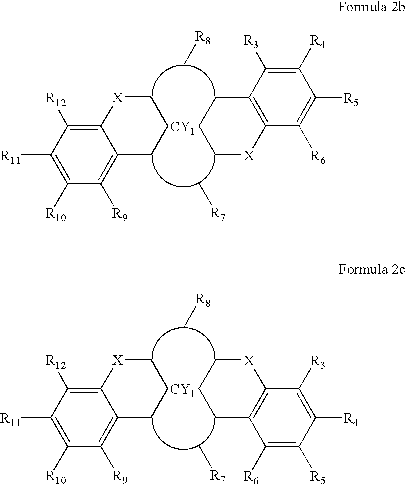

wherein X is independently C(R₁)(R₂), Si(R₁₃)(R₁₄), or Ge(R₁₃)(R₁₄) where R₁ and R₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, R₁ and R₂ may be connected to form a substituted or unsubstituted C₅-C₆₀ aromatic ring or a substituted or unsubstituted C₅-C₆₀ aliphatic ring, R₁₃ and R₁₄ are each independently a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, and R₁₃ and R₁₄ may be connected to form a substituted or unsubstituted C₅-C₆₀ aromatic ring or a substituted or unsubstituted C₅-C₆₀ aliphatic ring;

R₃, R₄, R₅, R₆, R₇, R₈, R₉, R₁₀, R₁₁, and R₁₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₆₀ arylamino group, or a substituted or unsubstituted C₁-C₆₀ alkylamino group, and two or more selected from R₃, R₄, R₅, R₆, R₇, R₈, R₉, R₁₀, R₁₁, and R₁₂ may be connected to form a fused substituted or unsubstituted C₆-C₆₀ aromatic ring or a fused substituted or unsubstituted C₆-C₆₀ heteroaromatic ring; and

CY1 is a substituted or unsubstituted C₅-C₆₀ aromatic ring or a substituted or unsubstituted C₂-C₆₀ heteroaromatic ring.

In more detail, CY1 may be selected from the group consisting of a pentalene ring, an indene ring, a naphthalene ring, an anthracene ring, an azulene ring, a heptalene ring, an acenaphthylene ring, a phenalene ring, a fluorene ring, a phenanthrene ring, a tetracene ring, a triphenylene ring, a pyrene ring, a chrysene ring, an ethyl-chrysene ring, a picene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, an indole ring, a benzimidazole ring, a quinoline ring, a benzothiophene ring, a parathiazine ring, a thianthrene ring, a fluoranthene ring, a benzofluoranthene ring, and derivatives thereof.

In the above formulae, the “aryl group” refers to a monovalent group having an aromatic ring system and may contain two or more ring systems. The two or more ring systems may be attached to each other or may be fused. The “heteroaryl group” refers to an aryl group in which at least one carbon atom is substituted by at least one atom selected from the group consisting of N, O, S, and P. The “cycloalkyl group” refers to an alkyl group having a ring system, and the “heterocycloalkyl group” refers to a cycloalkyl group in which at least one carbon atom is substituted by at least one atom selected from the group consisting of N, O, S, and P. The “fused aromatic ring or fused heteroaromatic ring” is present in a fused form with a backbone of Formula 1, 2a, 2b, or 2c, and may contain two or more ring systems. The two or more ring systems may be attached to each other or may be fused. The “heteroaromatic ring” refers to an aromatic ring in which at least one carbon atoms is substituted by at least one atom selected from the group consisting of N, O, S, and P.

The alkyl group, the alkenyl group, the alkynyl group, the cycloalkyl group, the cycloalkenyl group, the cycloalkynyl group, the aryl group, the heteroaryl group, the arylamino group, the alkylamino group, the aliphatic ring, the aromatic ring, and the heteroaromatic ring may be substituted by at least one substituent selected from the group consisting of —F; —Cl; —Br; —CN; —NO₂; —OH; a C₁-C₆₀ alkyl group which is unsubstituted or substituted by —F, —Cl, —Br, —CN, —NO₂, or —OH; a C₅-C₆₀ cycloalkyl group which is unsubstituted or substituted by a C₁-C₆₀ alkyl group, —F, —Cl, —Br, —CN, —NO₂, or —OH; a C₅-C₆₀ aryl group which is unsubstituted or substituted by a C₁-C₆₀ alkyl group, —F, —Cl, —Br, —CN, —NO₂, or —OH; and a C₂-C₆₀ heteroaryl group which is unsubstituted or substituted by a C₁-C₆₀ alkyl group, —F, —Cl, —Br, —CN, —NO₂, or —OH.

In more detail, R₁, R₂, R₃, R₄, R₅, R₆, R₇, R₈, R₉, R₁₀, R₁₁, R₁₂, R₁₃, and R₁₄ may be each independently selected from the group consisting of a C₁-C₆₀ alkyl group, a C₂-C₆₀ alkenyl group, a C₂-C₆₀ alkynyl group, a C₅-C₆₀ cycloalkyl group, a C₅-C₆₀ cycloalkenyl group, a C₅-C₆₀ cycloalkynyl group, a cyclohexyl group, a phenyl group, a biphenyl group, a pentalenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpholinyl group, a di(C₅-C₆₀ aryl)amino group, a tri(C₅-C₆₀ aryl)silyl group, a diphenylaminophenyl group, a ditolylaminophenyl group, and derivatives thereof. As used herein, the term “derivative(s)” refers to the above-illustrated group(s) wherein at least one hydrogen is substituted by at least one of the above-described substituents. Among the above-described groups, a methyl group, a cyclohexyl group, a phenyl group, a biphenyl group, a tolyl group, a naphthyl group, a pyrenyl group, a phenanthrenyl group, a fluorenyl group, an imidazolinyl group, an indolyl group, a quinolinyl group, a diphenylamino group, a N,N-diphenylaminophenyl group, a N,N-di-p-tolylaminophenyl group, and a triphenylsilyl group are preferred. Further, X may be CH₂, C(CH₃)₂, C(C₆H₅)₂, or C(C₆H₁₁)₂.

In more detail, according to an embodiment, examples of the organic light-emitting compound include, but are not limited to, compounds represented by Formulae 3 through 28 below:

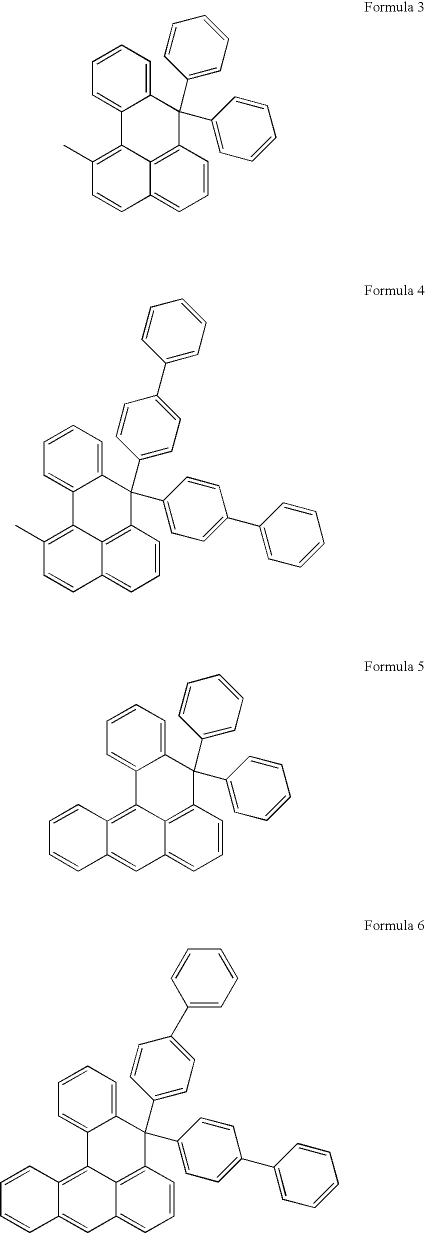

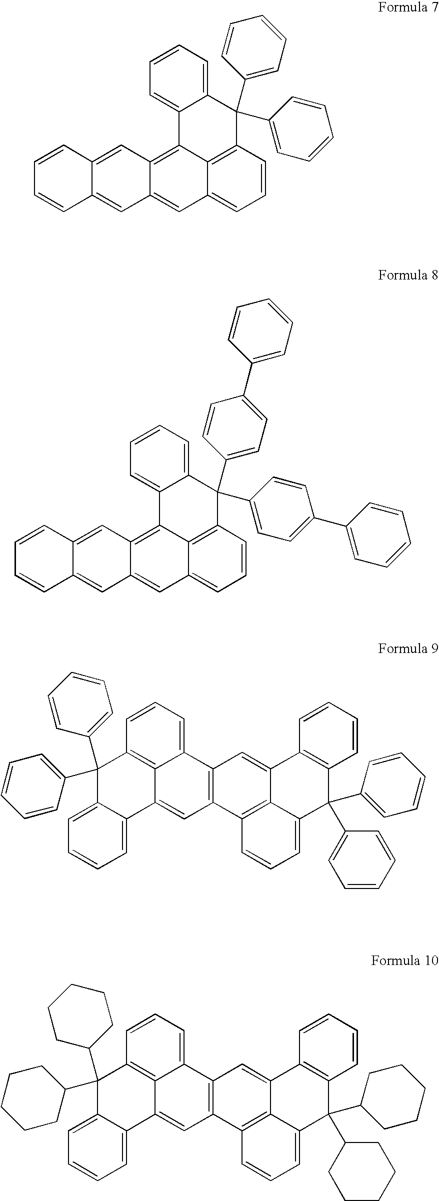

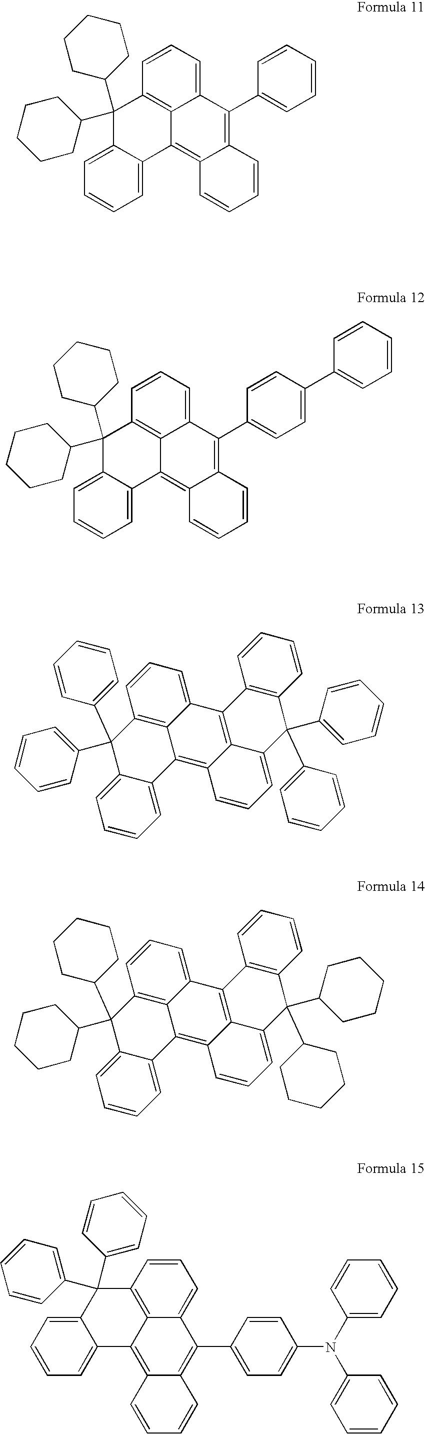

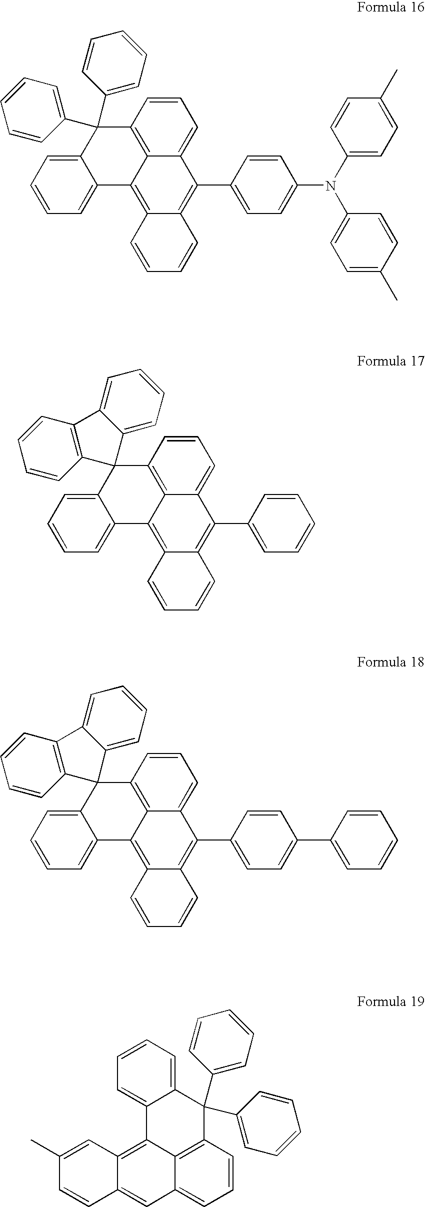

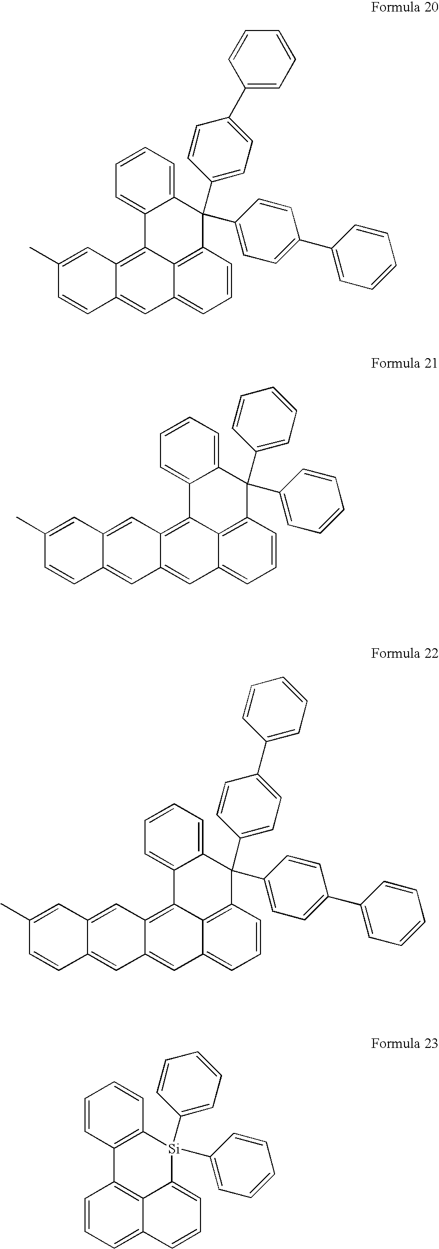

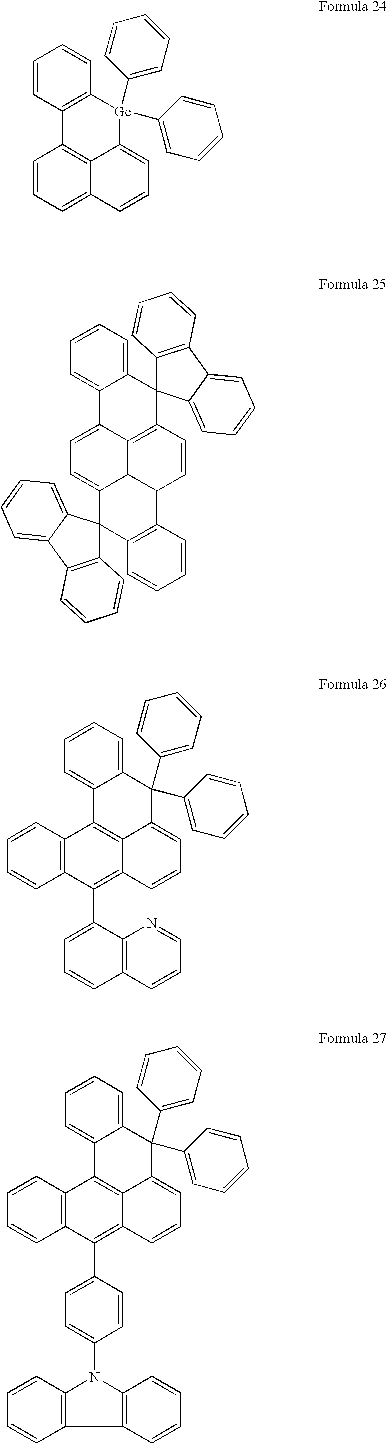

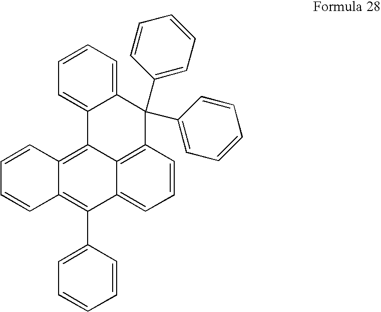

The compounds of Formulae 1, 2a, 2b, and 2c can be synthesized using a conventional synthesis method. For detailed synthesis procedures for these compounds, a reference may be made to the reaction schemes in the following synthesis examples.

Among compounds represented by Formula 2a, compounds including Si or Ge can be prepared according to Reaction Scheme 1a below:

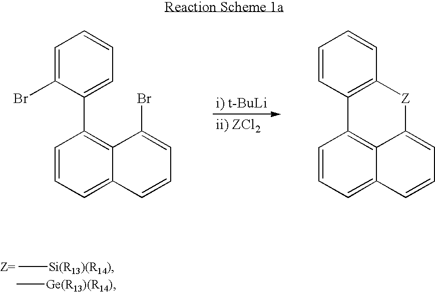

That is, the compounds of Formulae 23 and 24 can be obtained by replacing two bromo groups of 1-(2-bromophenyl)-8-bromonaphthalene with lithium and reacting the resultant products with ZCl₂.

The thermal stability of the above-described compounds can be evaluated by measuring the glass transition temperatures (Tg) and melting points (Tm) of the compounds through thermal analyses using Thermo Gravimetric Analysis (“TGA”) and Differential Scanning Calorimetry (“DSC”). For example, the compound of Formula 5 has Tg of 100° C. and Tm of 226° C., and the compound of Formula 28 has Tg of 137° C. and Tm of 324° C. The results show that organic light-emitting compounds according to the present invention have good thermal stability.

The present invention also provides an organic light-emitting device including:

a first electrode;

a second electrode; and

an organic layer interposed between the first electrode and the second electrode, the organic layer including at least one selected from compounds represented by Formulae 1 through 28 above.

The compound of Formula 1 above is suitable to be used for an organic layer of an organic light-emitting device, in particular, an emitting layer, a hole injection layer, a hole blocking layer, an electron transport layer, or a hole transport layer.

The organic light-emitting device includes an organic light-emitting compound that has good solubility and thermal stability and can form a stable organic layer, and thus, can show a low operating voltage and enhanced emission characteristics (such as, for example, color purity), unlike a conventional organic light-emitting device including a less stable organic layer when manufactured using a solution coating process. The organic light-emitting device can be variously structured. At least one layer selected from the group consisting of a hole injection layer, a hole transport layer, a hole blocking layer, an electron blocking layer, an electron transport layer, and an electron injection layer may be further interposed between the first electrode and the second electrode.

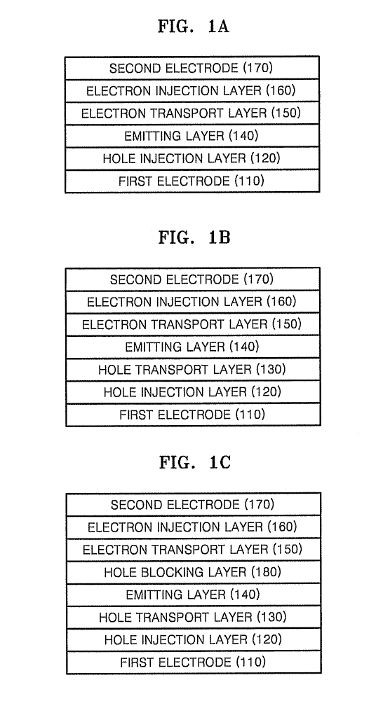

In more detail, organic light-emitting devices according to some embodiments are illustrated in FIGS. 1A, 1B, and 1C. Referring to FIG. 1A, an organic light-emitting device has a stacked (i.e., layered) structure comprising a first electrode 110/hole injection layer 120/emitting layer 140/electron transport layer 150/electron injection layer 160/second electrode 170. Referring to FIG. 1B, an organic light-emitting device has a stacked structure comprising a first electrode 110/hole injection layer 120/hole transport layer 130/emitting layer 140/electron transport layer 150/electron injection layer 160/second electrode 170. Referring to FIG. 1C, an organic light-emitting device has a stacked structure comprising a first electrode 110/hole injection layer 120/hole transport layer 130/emitting layer 140/hole blocking layer 180/electron transport layer 150/electron injection layer 160/second electrode 170. Here, at least one of the emitting layer 140, the hole injection layer 120, and the hole transport layer 130 may include an organic light-emitting compound as disclosed herein. An emitting layer 140 of the organic light-emitting device may include a red, green, blue, or white phosphorescent or fluorescent dopant. The phosphorescent dopant may be an organometallic compound including at least one element selected from the group consisting of Ir, Pt, Os, Ti, Zr, Hf, Eu, Tb, and Tm.

Hereinafter, a method of manufacturing an organic light-emitting device will be described with reference to FIG. 1C.

First, a first electrode material with a high work function is formed on a substrate (not shown) using deposition or sputtering to form a first electrode 110. The first electrode 110 may be an anode. Here, the substrate may be a substrate commonly used in organic light-emitting devices. Preferably, the substrate is a glass or transparent plastic substrate which is excellent in mechanical strength, thermal stability, transparency, surface smoothness, handling property, and water repellency. The first electrode material may be a material with transparency and good conductivity, e.g., indium tin oxide (“ITO”), indium zinc oxide (“IZO”), tin oxide (“SnO₂”), or zinc oxide (“ZnO”).

Next, a hole injection layer 120 (“HIL”) may be formed on a surface of the first electrode 110 opposite the substrate using various methods such as vacuum deposition, spin-coating, casting, or Langmuir-Blodgett (“LB”) film method. In the case of forming the hole injection layer 120 using a vacuum deposition process, the deposition conditions vary according to the type of a hole injection layer material, the structure and thermal characteristics of the hole injection layer 120, and the like. However, it is preferred that the hole injection layer 120 is deposited to a thickness of about 10 Å to about 5 μm at a deposition rate of about 0.01 to about 100 Å/sec, at a temperature of about 100 to about 500° C., at a vacuum level of about 10⁻⁸ to about 10⁻³ torr.

In the case of forming the hole injection layer 120 using a spin-coating process, the coating conditions vary according to the type of a hole injection layer material, the structure and thermal characteristics of the hole injection layer 120, and like considerations. However, it is preferred that the spin-coating is performed at a coating speed of about 2,000 to about 5,000 rpm, and, after spin-coating, a thermal treatment is performed at a temperature of about 80 to about 200° C. for the purpose of solvent removal.

The hole injection layer material may be a compound of Formula 1 as described above. In addition, the hole injection layer material may be a known hole injection material, such as, for example, a phthalocyanine compound (such as, for example, copper phthalocyanine) as disclosed in U.S. Pat. No. 4,356,429, a Starburst-type amine derivative (such as, for example, 4,4′,4″-tri(N-carbazolyl) triphenylamine (“TCTA”), 4,4′,4″-tri(N-3-methylphenyl-N-phenylamino) triphenylamine (“m-MTDATA”), or 4,4′,4″-tris[4-(3-methylphenylphenylamino)phenyl] benzene (“m-MTDAPB”) disclosed in Advanced Materials 1994, vol. 6, p. 677, or a soluble conductive polymer, such as, for example, polyaniline/dodecylbenzenesulfonic acid (“Pani/DBSA”), poly(3,4-ethylenedioxythiophene)/poly(4-styrenesulfonate) (“PEDOT/PSS”), polyaniline/camphor sulfonic acid (“Pani/CSA”), or polyaniline/poly(4-styrenesulfonate) (“PANI/PSS”).

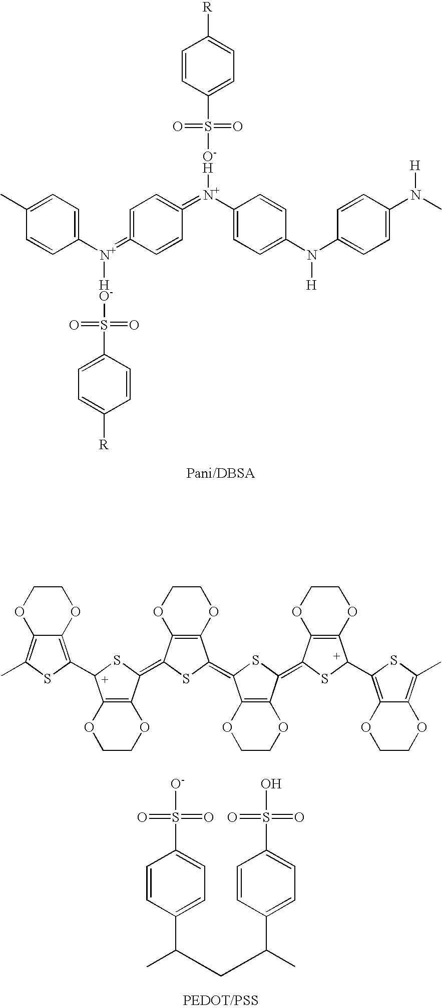

The hole injection layer 120 may be formed to a thickness of about 100 to about 10,000 Å, preferably about 100 to about 1,000 Å. If the thickness of the hole injection layer 120 is less than about 100 Å, the hole injection characteristics of the layer may be reduced. On the other hand, if the thickness of the hole injection layer exceeds about 10,000 Å, the operating voltage of the OLED may increase.

Next, a hole transport layer (“HTL”) 130 may be formed on a surface of the hole injection layer 120 opposite the first electrode 110 using any of a number of various methods such as, for example, vacuum deposition, spin-coating, casting, or an LB method. In the case of forming the hole transport layer 130 using vacuum deposition or spin-coating, the deposition or coating conditions vary according to the type of a compound used, but are generally similar to those conditions used for the formation of the hole injection layer 120.

A hole transport layer material may be a compound of Formula 1 as described above. In addition, the hole transport layer material can be a known hole transport material, such as, for example, a carbazole derivative such as N-phenylcarbazole or polyvinylcarbazole; an amine derivative having an aromatic fused ring such as N,N′-bis(3-methylphenyl)-N,N′-diphenyl-[1,1-biphenyl]-4,4′-diamine (“TPD”) or N,N′-di(naphthalene-1-yl)-N,N′-diphenylbenzidine (“α-NPD”); or the like. The hole transport layer 130 may be formed to a thickness of about 50 to about 1,000 Å, preferably about 100 to about 600 Å. If the thickness of the hole transport layer 130 is less than about 50 Å, the hole transport characteristics of the layer may be reduced. On the other hand, if the thickness of the hole transport layer exceeds about 1,000 Å, the operating voltage of the OLED may increase.

Next, an emitting layer (“EML”) 140 may be formed on a surface of the hole transport layer 130 opposite the HIL 120 using a suitable method such as, for example, vacuum deposition, spin-coating, casting, or LB method. In the case of forming the emitting layer 140 using vacuum deposition or spin-coating, the deposition or coating conditions can vary according to the type of compound used, but are generally similar to those conditions used for the formation of the hole injection layer 120.

The emitting layer 140 can include a compound of Formula 1 as described above. Here, a known host or dopant material suitable for use with the compound of Formula 1 may also be included. The compound of Formula 1 may also be used alone. For example, the host material may be tris(8-quinolinolate)aluminum (“Alq3”), 4,4′-N,N′-dicarbazole-biphenyl (“CBP”), poly(n-vinylcarbazole) (“PVK”), or the like.

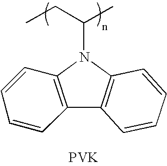

For the dopant material, fluorescent or phosphorescent materials may be used. An exemplary fluorescent dopant can be IDE102 or IDE105 (commercially available from Idemitsu), C545T (commercially available from Hayashibara), and the like. An exemplary phosphorescent dopant can be a red phosphorescent dopant (such as, for example, platinum octatethyl porphyrin (“PtOEP”)(RD 61, available from UDC)), a green phosphorescent dopant (such as, for example, Ir(PPy)3 (PPy=2-phenylpyridine)), or a blue phosphorescent dopant (such as, for example, iridium (III) bis[4,6-di-fluorophenyl)-pyridinato-N,C²′]picolinate (referred to herein as both “FIrpic” and “F₂Irpic”).

The doping concentration of a dopant is not particularly limited. Generally, the content of dopant is 0.01 to 15 parts by weight based on 100 parts by weight of host.

The emitting layer 140 may be formed to a thickness of about 100 to about 1,000 Å, preferably about 200 to about 600 Å. If the thickness of the emitting layer 140 is less than about 100 Å, the emission characteristics of the layer may be reduced. On the other hand, if the thickness of the emitting layer 140 exceeds about 1,000 Å, the operating voltage of the OLED may increase.

In a case where the emitting layer 140 includes a phosphorescent dopant, a hole blocking layer (“HBL”) 180 can be formed on a surface of the hole transport layer 130 opposite using a suitable method such as, for example, vacuum deposition, spin-coating, casting, or LB method, in order to prevent the diffusion of triplet excitons or holes into the electron transport layer 150. In the case of forming the hole blocking layer 180 using vacuum deposition or spin coating, the deposition or coating conditions vary according to the type of compound used, but are generally almost similar to those conditions used for the formation of the hole injection layer 120. An available hole blocking material may be an oxadiazole derivative, a triazole derivative, a phenanthroline derivative, BCP, a hole blocking material as disclosed in Japanese Patent Laid-Open Publication No. Hei. 11-329734, and the like. The hole blocking layer 180 may be formed to a thickness of about 50 to about 1,000 Å, preferably about 100 to about 300 Å. If the thickness of the hole blocking layer is less than about 50 Å, the hole blocking characteristics may be reduced. On the other hand, if the thickness of the hole blocking layer exceeds about 1,000 Å, the operating voltage of the OLED may increase.

Next, an electron transport layer (“ETL”) 150 may be formed using any of a variety of methods such as for example vacuum deposition, spin-coating, or casting. In the case of forming the electron transport layer 150 using vacuum deposition or spin-coating, the deposition or coating conditions can vary according to the type of compound used, but are generally similar to those conditions used for the formation of the hole injection layer 120. An electron transport layer material serves to stably transport electrons from an electron donor electrode (a cathode) and may be a known material such as a quinoline derivative, in particular, Alq3, TAZ (see below), or bis(2-methyl-8-quinolinolato)-aluminum biphenolate (“Balq”).

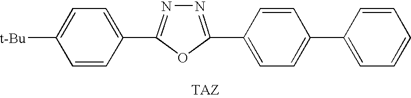

The electron transport layer 150 may be formed to a thickness of about 100 to about 1,000 Å, preferably about 200 to about 500 Å. If the thickness of the electron transport layer is less than about 100 Å, the electron transport characteristics may be reduced. On the other hand, if the thickness of the electron transport layer exceeds about 1,000 Å, the operating voltage of the OLED may increase.

An electron injection layer (“EIL”) 160 may be formed on a surface of the electron transport layer 150 opposite the hole blocking layer 180, in order to facilitate the injection of electrons from a cathode into the electron transport layer 150. The electron injection layer material is not particularly limited.

The electron injection layer 160 material may, where used, be selected from known materials such as LiF, NaCl, CsF, Li₂O, or BaO. The deposition conditions of the electron injection layer 160 can vary according to the compound used, but are generally similar to those conditions used for the formation of the hole injection layer 120.

The electron injection layer 160 may be formed to a thickness of about 1 to about 100 Å, preferably about 5 to about 50 Å. If the thickness of the electron injection layer is less than about 1 Å, the electron injection characteristics may be reduced. On the other hand, if the thickness of the electron injection layer 160 exceeds about 100 Å, the operating voltage of the OLED may increase.

Finally, a second electrode 170 may be formed on a surface of the electron injection layer 160 opposite the electron transport layer 150 using a suitable method such as, for example, vacuum deposition or sputtering. The second electrode may be used as a cathode. A material for forming the second electrode 170 may be metal or metal alloy with a low work function, an electroconductive compound, or a mixture thereof. For example, the second electrode material may be lithium (Li), magnesium (Mg), aluminum (Al), aluminum-lithium (Al—Li), calcium (Ca), magnesium-indium (Mg—In), magnesium-silver (Mg—Ag), or the like. The second electrode 170 may also be a transmissive cathode formed of ITO or IZO to provide a front-emission type device.

The present invention also provides a method of manufacturing an organic light-emitting device, the method including: forming a first electrode; forming on a surface of the first electrode an organic layer including a compound selected from compounds represented by Formulae 1 through 28; and forming a second electrode on a surface of the organic layer opposite the first electrode. In particular, the organic layer may be formed using a dry- or wet-spray process, such as, for example, vacuum deposition, spin-coating, inkjet printing, or spray printing, or a thermal transfer process.

Hereinafter, the present invention will be described more specifically with reference to the following working examples. However, the following examples are only for illustrative purposes and are not intended to limit the scope of the invention.

In the following working examples, compounds corresponding in structure to an above described formula X (where X represents the Formula number 5, 13, or 28) will be referred to hereinbelow as “compound X” (for example, a compound represented by Formula 5 will be referred to as “compound 5”). All synthesized compounds were identified by ¹H NMR spectroscopy and mass spectrometry.

### EXAMPLES

### Synthesis Example 1

A compound 5 (corresponding to Formula 5, above) was synthesized according to Reaction Schemes 1 and 2 below.

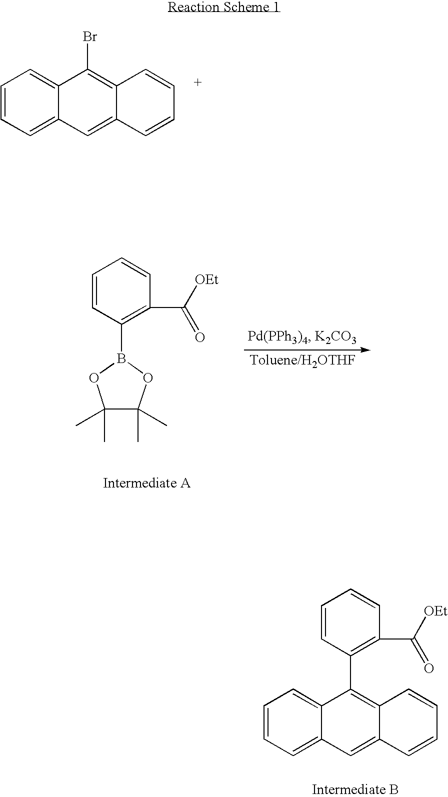

### Synthesis of Intermediate B

0.55 g (2.2 mmol) of 9-bromoanthracene was dissolved in THF (5 ml). Then, a solution of 0.6 g (2.2 mmol) of an intermediate A, 75 mg (0.06 mmol) of tetrakis triphenylphosphine palladium (Pd(PPh₃)₄), and 298 mg (2.2 mmol) of potassium carbonate (K₂CO₃) in 5 ml of toluene and 2.5 ml of water was added thereto, and the reaction mixture was refluxed for 24 hours. After the reaction was terminated, a solvent was removed by evaporation, and the residue was washed with 100 ml of ethylacetate and 100 ml of water. The organic layer was collected and dried over anhydrous magnesium sulfate. The crude product was purified by silica chromatography to give 0.20 g (yield: 27%) of an intermediate B.

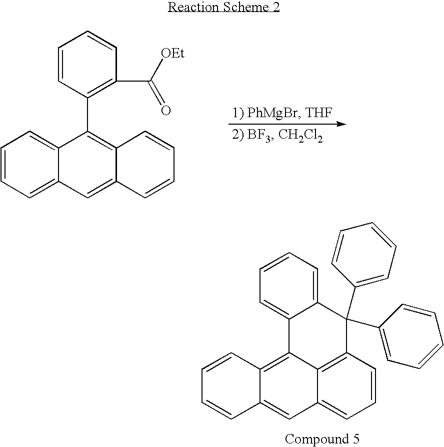

### Synthesis of Compound 5

1.4 g (4.2 mmol) of the intermediate B was dissolved in THF (33 ml), and phenyl magnesium bromide (PhMgBr 1.0 M, 9 ml) was added thereto. The reaction mixture was heated to 70° C. and stirred for one hour. After the reaction was terminated, the resultant solution was washed with 100 ml of water and 100 ml of ethyl acetate. The organic layer was collected, and dried over anhydrous magnesium sulfate and then under a reduced pressure. The resultant solid was dissolved in methylene chloride (42 ml), and trifluorinated boron (0.5 ml) was added thereto. The reaction mixture was stirred for 30 minutes, and methanol (2 ml) was added thereto so that the reaction was terminated. The resultant solution was washed with 200 ml of methylene chloride and 200 ml of water, and the organic layer was collected and dried over anhydrous magnesium sulfate. The crude product was purified by silica chromatography to give 1.1 g (yield: 57%) of the compound 5.

¹H-NMR (CDCl₃, 300 MHz, ppm): 8.6-6.9 (m, 22H).

### Synthesis Example 2

A compound 13 (corresponding to Formula 13, above) was synthesized according to Reaction Schemes 3 and 4 below.

### Synthesis of Intermediate C

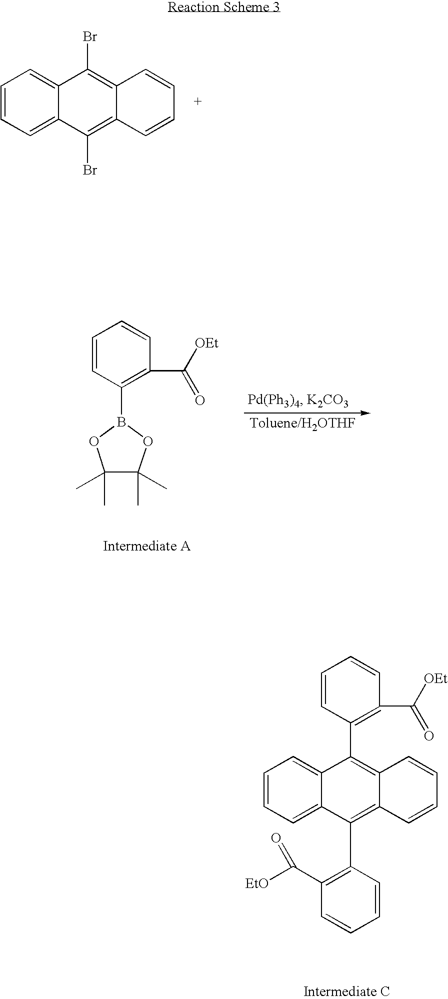

0.73 g (2.2 mmol) of 9,10-dibromoanthracene was dissolved in THF (5 ml). Then, a solution of 0.6 g (2.2 mmol) of an intermediate A, 75 mg (0.06 mmol) of tetrakis triphenylphosphine palladium (Pd(PPh₃)₄), and 298 mg (2.2 mmol) of potassium carbonate (K₂CO₃) in 5 ml of toluene and 2.5 ml of water was added thereto, and the reaction mixture was refluxed for 24 hours. After the reaction was terminated, the solvent was removed by evaporation. The residue was washed with 100 ml of ethyl acetate and 100 ml of water, and the organic layer was collected and dried over anhydrous magnesium sulfate. The crude product was purified by silica chromatography to give 0.45 g (yield: 43%) of an intermediate C.

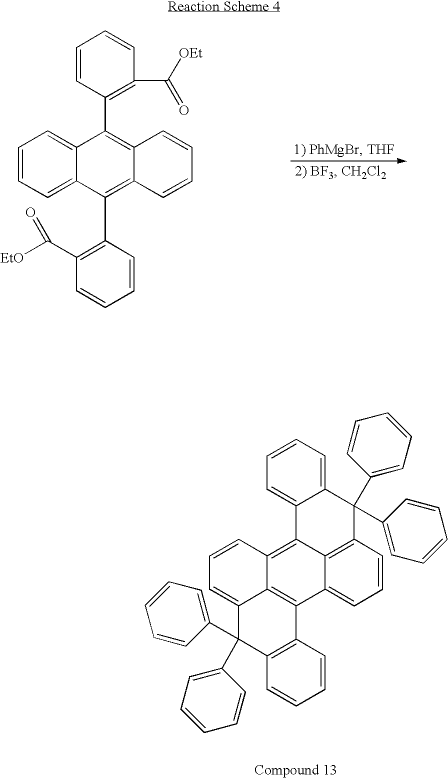

### Synthesis of Compound 13

1.0 g (2.1 mmol) of the intermediate C was dissolved in THF (15 ml), and phenyl magnesium bromide (PhMgBr 1.0 M, 4.5 ml) was added thereto. The reaction mixture was heated to 70° C. and stirred for one hour. After the reaction was terminated, 50 ml of water and 50 ml of ethyl acetate were added thereto. The organic layer was collected, and dried over anhydrous magnesium sulfate and the solvent removed under reduced pressure. The resultant solid was dissolved in methylene chloride (21 ml), and trifluorinated boron (0.5 ml) was added thereto. The reaction mixture was stirred for 30 minutes, and methanol (1 ml) was added thereto so that the reaction was terminated. The resultant solution was washed with 100 ml of methylene chloride and 100 ml of water, and the organic layer was collected and dried over anhydrous magnesium sulfate. The crude product was purified by silica chromatography to give 0.7 g (yield: 48%) of the compound 13.

¹H-NMR (CDCl₃, 300 MHz, ppm): 8.7-6.9 (m, 34H).

### Synthesis Example 3

A compound 28 (corresponding to Formula 28 above) was synthesized according to Reaction Schemes 5 and 6 below.

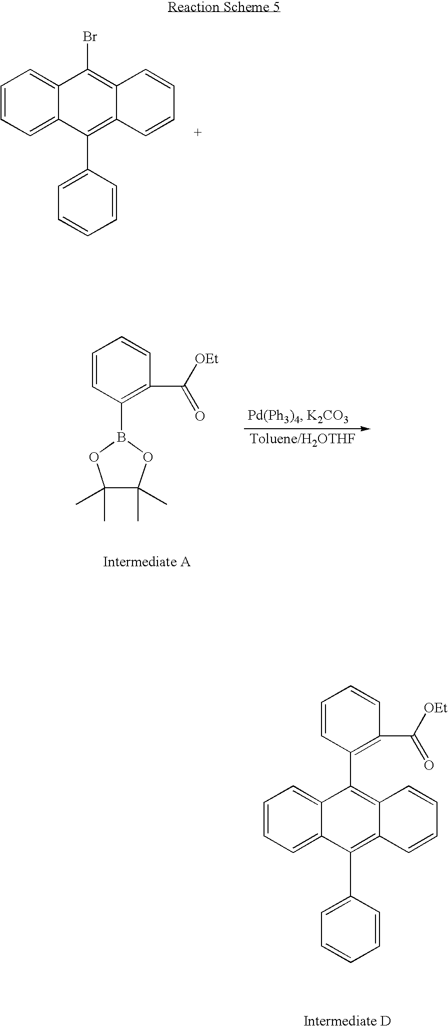

### Synthesis of Intermediate D

1.5 g (4.4 mmol) of 9-bromo-10-phenyl anthracene was dissolved in THF (10 ml). Then, a solution of 1.2 g (4.4 mmol) of an intermediate A, 150 mg (0.12 mmol) of tetrakis triphenylphosphine palladium (Pd(PPh₃)₄), and 600 mg (4.4 mmol) of potassium carbonate (K₂CO₃) in 10 ml of toluene and 5 ml of water was added thereto and the reaction mixture was refluxed for 24 hours. After the reaction was terminated, solvent was removed by evaporation. The residue was washed with 200 ml of ethylacetate and 200 ml of water, and the organic layer was collected and dried over anhydrous magnesium sulfate. The crude product was purified by silica chromatography to give 0.85 g (yield: 39%) of an intermediate D.

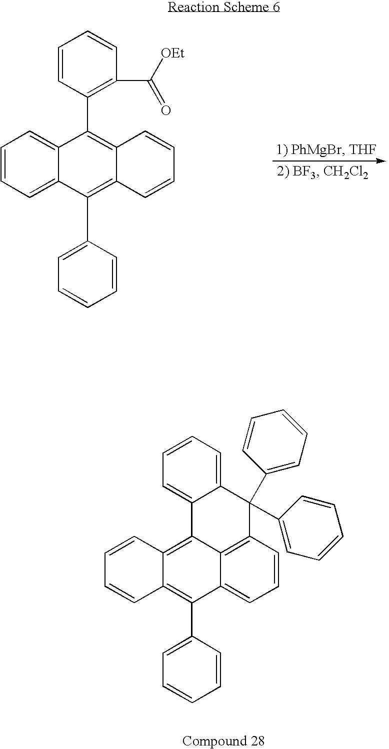

### Synthesis of Compound 28

1.1 g (2.9 mmol) of the intermediate D was dissolved in THF (18 ml), and phenyl magnesium bromide (PhMgBr 1.0 M, 6 ml) was added thereto. The reaction mixture was heated to 70° C. and stirred for one hour. After the reaction was terminated, the resultant solution was washed with 100 ml of water and 100 ml of ethyl acetate. The organic layer was collected, and dried over anhydrous magnesium sulfate and then concentrated under reduced pressure. The resultant solid was dissolved in methylene chloride (27 ml), and trifluorinated boron (0.3 ml) was added thereto. The reaction mixture was stirred for 30 minutes, and methanol (2 ml) was added thereto so that the reaction was terminated. The resultant solution was washed with 200 ml of methylenechloride and 200 ml of water, and the organic layer was collected and dried over anhydrous magnesium sulfate. The crude product was purified by silica chromatography to give 0.9 g (yield: 62%) of the compound 28.

¹H-NMR (CDCl₃, 300 MHz, ppm): 8.7-6.9 (m, 26H).

### Evaluation Example 1: Evaluation of Emission Characteristics of Compounds (In Solution Phase)

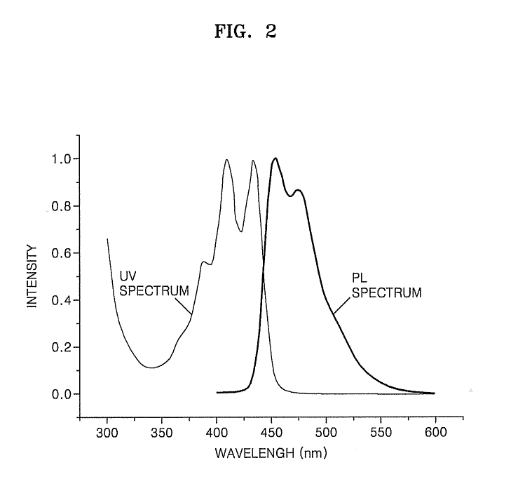

Emission characteristics of the compounds 5, 13, and 28 were evaluated by measuring the UV and PL (photoluminescence) spectra of the compounds 5, 13, and 28. First, the compound 5 was diluted with toluene to a concentration of 0.2 mM, and the UV absorption spectrum of the diluted solution was measured using a Shimadzu UV-350 spectrometer. The same experiment was performed for the compounds 13 and 28. Meanwhile, the compound 5 was diluted with toluene to a concentration of 10 mM, and the PL spectrum of the compound 5 was measured using a ISC PC1 spectrofluorometer equipped with a Xenon lamp. The results are presented in Table 1 below. The same experiment was performed for the compounds 13 and 28. The UV and PL spectra of the compound 5 are illustrated in FIG. 2.

TABLE 1

| | Maximum UV absorption | Maximum PL |
|---|---|---|
| Compound | wavelength (nm) | wavelength (nm) |
| 5 | 410 | 455 |
| 13 | 430 | 490 |
| 28 | 420 | 470 |

The above results show that a compound according to the present invention has emission characteristics suitable to be used in organic light-emitting devices.

### Example 1

Organic light-emitting devices having the following structure were manufactured using the compound 5 as a dopant of an emitting layer and 9,10-di(naphthalene-2-yl)anthracene (“ADN”) represented by Formula 29 below as a host of the emitting layer: ITO/α-NPD (300 Å)/compound 5+ADN (300 Å)/Alq3 (200 Å)/LiF (10 Å)/Al (2,000 Å). A 15Ω/cm² (1,000 Å) ITO glass substrate was cut into pieces of 50 mm×50 mm×0.7 mm in size, followed by ultrasonic cleaning in acetone, isopropyl alcohol, and pure water (15 minutes for each) and then UV/ozone cleaning (30 minutes) to form anodes. Then, α-NPD was vacuum-deposited to a thickness of 300 Å on the anodes at a deposition rate of 1 Å/sec to form hole transport layers. Then, the compound 5 and ADN were vacuum-deposited on the hole transport layers at a deposition rate of 5 Å/sec and 30 Å/sec, respectively, to form emitting layers with a thickness of 300 Å. Then, an Alq3 compound was vacuum-deposited to a thickness of 200 Å on the emitting layers to form electron transport layers. LiF (10 Å, electron injection layers) and Al (2,000 Å, cathodes) were sequentially vacuum-deposited on the electron transport layers to thereby complete organic light-emitting devices as illustrated in FIG. 1A. The organic light-emitting devices were designated “sample 1”.

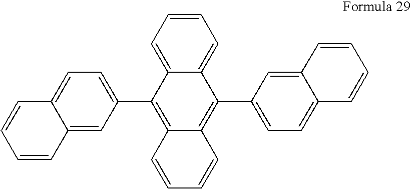

### Examples 2 and 3

Organic light-emitting devices were manufactured in the same manner as in Example 1 using the compounds 13 and 28, and were designated “samples 2 and 3”.

### Evaluation Example 2: Evaluation of Characteristics of Samples 1-3

For the samples 1-3, an operating voltage, brightness, and efficiency were evaluated using PR650 (Spectroscan) Source Measurement Unit. The results are presented in Table 2 below.

TABLE 2

| | Turn-on | Maximum efficiency | Maximum brightness |
|---|---|---|---|
| Sample | voltage (V) | (cd/A) | (cd/m²) |
| 1 | 3.4 | 4.3 | 7242 |
| 2 | 3.4 | 5.1 | 8700 |
| 3 | 3.4 | 4.7 | 7134 |

Table 2 shows that the samples 1-3 according to the present invention had excellent electrical characteristics.

A compound represented by Formula 1 according to the present invention has good solubility, and at the same time, good emission characteristics and thermal stability. Therefore, the use of the compound according to the present invention enables to produce an organic light-emitting device having a low operating voltage, and good brightness and efficiency.

What is claimed is:

1. An organic light-emitting compound represented by Formula 1, below: 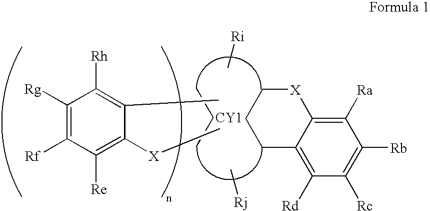
Wherein X is a C, Si, or Ge atom disubstituted with H or C₁₋₆₀ organic groups, Rₐ-Rⱼ are C₁₋₆₀ organic groups, and CY1 is a substituted or unsubstituted C₅-C₆₀ aromatic ring or a substituted or unsubstituted C₂-C₆₀ heteroaromatic ring.

2. The organic light-emitting compound of claim 1, represented by Formula 2a below: 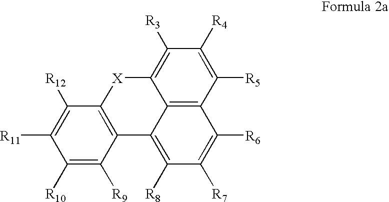
wherein X is C(R₁)(R₂), Si(R₁₃)(R₁₄), or Ge(R₁₃)(R₁₄) where R₁ and R₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, R₁ and R₂ may be connected to form a substituted or unsubstituted C₅-C₆₀ aromatic ring or a substituted or unsubstituted C₅-C₆₀ aliphatic ring, R₁₃ and R₁₄ are each independently a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, and R₁₃ and R₁₄ may be connected to form a substituted or unsubstituted C₅-C₆₀ aromatic ring or a substituted or unsubstituted C₅-C₆₀ aliphatic ring; and
R₃, R₄, R₅, R₆, R₇, R₈, R₉, R₁₀, R₁₁, and R₁₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₆₀ arylamino group, or a substituted or unsubstituted C₁-C₆₀ alkylamino group, and two or more selected from R₃, R₄, R₅, R₆, R₇, R₈, R₉, R₁₀, R₁₁, and R₁₂ may be connected to form a fused substituted or unsubstituted C₆-C₆₀ aromatic ring or a fused substituted or unsubstituted C₆-C₆₀ heteroaromatic ring.

3. The organic light-emitting compound of claim 2, wherein the alkyl group, the alkenyl group, the alkynyl group, the cycloalkyl group, the cycloalkenyl group, the cycloalkynyl group, the aryl group, the heteroaryl group, the arylamino group, the alkylamino group, the aliphatic ring, the aromatic ring, and the heteroaromatic ring are substituted by at least one selected from the group consisting of —F; —Cl; —Br; —CN; —NO₂; —OH; a C₁-C₆₀ alkyl group which is unsubstituted or substituted by —F, —Cl, —Br, —CN, —NO₂, or —OH; a C₅-C₆₀ cycloalkyl group which is unsubstituted or substituted by a C₁-C₆₀ alkyl group, —F, —Cl, —Br, —CN, —NO₂, or —OH; a C₅-C₆₀ aryl group which is unsubstituted or substituted by a C₁-C₆₀ alkyl group, —F, —Cl, —Br, —CN, —NO₂, or —OH; and a C₂-C₆₀ heteroaryl group which is unsubstituted or substituted by a C₁-C₆₀ alkyl group, —F, —Cl, —Br, —CN, —NO₂, or —OH.

4. The organic light-emitting compound of claim 2, wherein R₁, R₂, R₃, R₄, R₅, R₆, R₇, R₈, R₉, R₁₀, R₁₁, R₁₂, R₁₃, and R₁₄ are each independently selected from the group consisting of a C₁-C₆₀ alkyl group, a C₂-C₆₀ alkenyl group, a C₂-C₆₀ alkynyl group, a C₅-C₆₀ cycloalkyl group, a C₅-C₆₀ cycloalkenyl group, a C₅-C₆₀ cycloalkynyl group, a cyclohexyl group, a phenyl group, a biphenyl group, a pentalenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpholinyl group, a di(C₅-C₆₀ aryl)amino group, a tri(C₅-C₆₀ aryl)silyl group, a diphenylaminophenyl group, a ditolylaminophenyl group, and derivatives thereof.

5. The organic light-emitting compound of claim 2, wherein R₁, R₂, R₃, R₄, R₅, R₆, R₇, R₈, R₉, R₁₀, R₁₁, R₁₂, R₁₃, and R₁₄ are each independently selected from the group consisting of a methyl group, a cyclohexyl group, a phenyl group, a biphenyl group, a tolyl group, a naphthyl group, a pyrenyl group, a phenanthrenyl group, a fluorenyl group, an imidazolinyl group, an indolyl group, a quinolinyl group, a diphenylamino group, a N,N-diphenylaminophenyl group, a N,N-di-p-tolylaminophenyl group, a triphenylsilyl group, and derivatives thereof.

6. The organic light-emitting compound of claim 2, wherein X is CH₂, C(CH₃)₂, C(C₆H₅)₂, or C(C₆H₁₁)₂.

7. The organic light-emitting compound of claim 1, represented by Formula 2b below: 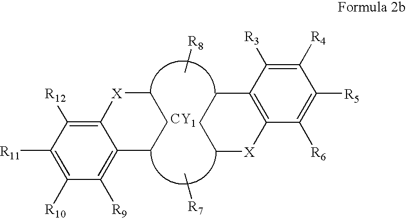
wherein X is C(R₁)(R₂), Si(R₁₃)(R₁₄), or Ge(R₁₃)(R₁₄) where R₁ and R₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, R₁ and R₂ may be connected to form a substituted or unsubstituted C₅-C₆₀ aromatic ring or a substituted or unsubstituted C₅-C₆₀ aliphatic ring, R₁₃ and R₁₄ are each independently a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, and R₁₃ and R₁₄ may be connected to form a substituted or unsubstituted C₅-C₆₀ aromatic ring or a substituted or unsubstituted C₅-C₆₀ aliphatic ring;
R₃, R₄, R₅, R₆, R₇, R₈, R₉, R₁₀, R₁₁, and R₁₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₆₀ arylamino group, or a substituted or unsubstituted C₁-C₆₀ alkylamino group, and two or more selected from R₃, R₄, R₅, R₆, R₇, R₈, R₉, R₁₀, R₁₁, and R₁₂ may be connected to form a fused substituted or unsubstituted C₆-C₆₀ aromatic ring or a fused substituted or unsubstituted C₆-C₆₀ heteroaromatic ring; and
CY1 is a substituted or unsubstituted C₅-C₆₀ aromatic ring or a substituted or unsubstituted C₂-C₆₀ heteroaromatic ring.

8. The organic light-emitting compound of claim 7, wherein CY1 is selected from the group consisting of a pentalene ring, an indene ring, a naphthalene ring, an anthracene ring, an azulene ring, a heptalene ring, an acenaphthylene ring, a phenalene ring, a fluorene ring, a phenanthrene ring, a tetracene ring, a triphenylene ring, a pyrene ring, a chrysene ring, an ethyl-chrysene ring, a picene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, an indole ring, a benzimidazole ring, a quinoline ring, a benzothiophene ring, a parathiazine ring, a thianthrene ring, a fluoranthene ring, a benzofluoranthene ring, and derivatives thereof.

9. The organic light-emitting compound of claim 7, wherein the alkyl group, the alkenyl group, the alkynyl group, the cycloalkyl group, the cycloalkenyl group, the cycloalkynyl group, the aryl group, the heteroaryl group, the arylamino group, the alkylamino group, the aliphatic ring, the aromatic ring, and the heteroaromatic ring are substituted by at least one selected from the group consisting of —F; —Cl; —Br; —CN; —NO₂; —OH; a C₁-C₆₀ alkyl group which is unsubstituted or substituted by —F, —Cl, —Br, —CN, —NO₂, or —OH; a C₅-C₆₀ cycloalkyl group which is unsubstituted or substituted by a C₁-C₆₀ alkyl group, —F, —Cl, —Br, —CN, —NO₂, or —OH; a C₅-C₆₀ aryl group which is unsubstituted or substituted by a C₁-C₆₀ alkyl group, —F, —Cl, —Br, —CN, —NO₂, or —OH; and a C₂-C₆₀ heteroaryl group which is unsubstituted or substituted by a C₁-C₆₀ alkyl group, —F, —Cl, —Br, —CN, —NO₂, or —OH.

10. The organic light-emitting compound of claim 7, wherein R₁, R₂, R₃, R₄, R₅, R₆, R₇, R₈, R₉, R₁₀, R₁₁, R₁₂, R₁₃, and R₁₄ are each independently selected from the group consisting of a C₁-C₆₀ alkyl group, a C₂-C₆₀ alkenyl group, a C₂-C₆₀ alkynyl group, a C₅-C₆₀ cycloalkyl group, a C₅-C₆₀ cycloalkenyl group, a C₅-C₆₀ cycloalkynyl group, a cyclohexyl group, a phenyl group, a biphenyl group, a pentalenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpholinyl group, a di(C₅-C₆₀ aryl)amino group, a tri(C₅-C₆₀ aryl)silyl group, a diphenylaminophenyl group, a ditolylaminophenyl group, and derivatives thereof.

11. The organic light-emitting compound of claim 7, wherein R₁, R₂, R₃, R₄, R₅, R₆, R₇, R₈, R₉, R₁₀, R₁₁, R₁₂, R₁₃, and R₁₄ are each independently selected from the group consisting of a methyl group, a cyclohexyl group, a phenyl group, a biphenyl group, a tolyl group, a naphthyl group, a pyrenyl group, a phenanthrenyl group, a fluorenyl group, an imidazolinyl group, an indolyl group, a quinolinyl group, a diphenylamino group, a N,N-diphenylaminophenyl group, a N,N-di-p-tolylaminophenyl group, a triphenylsilyl group, and derivatives thereof.

12. The organic light-emitting compound of claim 7, wherein X is CH₂, C(CH₃)₂, C(C₆H₅)₂, or C(C₆H₁₁)₂.

13. The organic light-emitting compound of claim 1, represented by Formula 2c below: 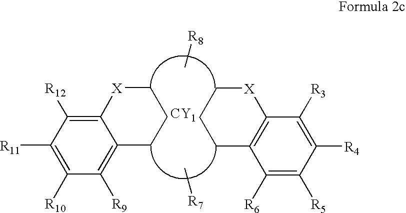
wherein X is C(R₁)(R₂), Si(R₁₃)(R₁₄), or Ge(R₁₃)(R₁₄) where R₁ and R₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, R₁ and R₂ may be connected to form a substituted or unsubstituted C₅-C₆₀ aromatic ring or a substituted or unsubstituted C₅-C₆₀ aliphatic ring, R₁₃ and R₁₄ are each independently a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₅₀ arylamino group, or a substituted or unsubstituted C₁-C₆₀ alkylamino group, and R₁₃ and R₁₄ may be connected to form a substituted or unsubstituted C₅-C₆₀ aromatic ring or a substituted or unsubstituted C₅-C₆₀ aliphatic ring;
R₃, R₄, R₅, R₆, R₇, R₈, R₉, R₁₀, R₁₁, and R₁₂ are each independently hydrogen, halogen, a cyano group, a nitro group, a hydroxyl group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₅-C₆₀ cycloalkyl group, a substituted or unsubstituted C₅-C₆₀ cycloalkenyl group, a substituted or unsubstituted C₅-C₆₀ cycloalkynyl group, a substituted or unsubstituted C₅-C₆₀ aryl group, a substituted or unsubstituted C₂-C₆₀ heteroaryl group, a substituted or unsubstituted C₅-C₆₀ arylamino group, or a substituted or unsubstituted C₁-C₆₀ alkylamino group, and two or more selected from R₃, R₄, R₅, R₆, R₇, R₈, R₉, R₁₀, R₁₁, and R₁₂ may be connected to form a fused substituted or unsubstituted C₆-C₆₀ aromatic ring or a fused substituted or unsubstituted C₆-C₆₀ heteroaromatic ring; and
CY1 is a substituted or unsubstituted C₅-C₆₀ aromatic ring or a substituted or unsubstituted C₂-C₆₀ heteroaromatic ring.

14. The organic light-emitting compound of claim 13, wherein CY1 is selected from the group consisting of a pentalene ring, an indene ring, a naphthalene ring, an anthracene ring, an azulene ring, a heptalene ring, an acenaphthylene ring, a phenalene ring, a fluorene ring, a phenanthrene ring, a tetracene ring, a triphenylene ring, a pyrene ring, a chrysene ring, an ethyl-chrysene ring, a picene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, an indole ring, a benzimidazole ring, a quinoline ring, a benzothiophene ring, a parathiazine ring, a thianthrene ring, a fluoranthene ring, a benzofluoranthene ring, and derivatives thereof.

15. The organic light-emitting compound of claim 13, wherein the alkyl group, the alkenyl group, the alkynyl group, the cycloalkyl group, the cycloalkenyl group, the cycloalkynyl group, the aryl group, the heteroaryl group, the arylamino group, the alkylamino group, the aliphatic ring, the aromatic ring, and the heteroaromatic ring are substituted by at least one selected from the group consisting of —F; —Cl; —Br; —CN; —NO₂; —OH; a C₁-C₆₀ alkyl group which is unsubstituted or substituted by —F, —Cl, —Br, —CN, —NO₂, or —OH; a C₅-C₆₀ cycloalkyl group which is unsubstituted or substituted by a C₁-C₆₀ alkyl group, —F, —Cl, —Br, —CN, —NO₂, or —OH; a C₅-C₆₀ aryl group which is unsubstituted or substituted by a C₁-C₆₀ alkyl group, —F, —Cl, —Br, —CN, —NO₂, or —OH; and a C₂-C₆₀ heteroaryl group which is unsubstituted or substituted by a C₁-C₆₀ alkyl group, —F, —Cl, —Br, —CN, —NO₂, or —OH.

16. The organic light-emitting compound of claim 13, wherein R₁, R₂, R₃, R₄, R₅, R₆, R₇, R₈, R₉, R₁₀, R₁₁, R₁₂, R₁₃, and R₁₄ are each independently selected from the group consisting of a C₁-C₆₀ alkyl group, a C₂-C₆₀ alkenyl group, a C₂-C₆₀ alkynyl group, a C₅-C₆₀ cycloalkyl group, a C₅-C₆₀ cycloalkenyl group, a C₅-C₆₀ cycloalkynyl group, a cyclohexyl group, a phenyl group, a biphenyl group, a pentalenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, an ovalenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolinyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxyranyl group, a pyrrolidinyl group, a pyrazolidinyl group, an imidazolidinyl group, a piperidinyl group, a piperazinyl group, a morpholinyl group, a di(C₅-C₆₀ aryl)amino group, a tri(C₅-C₆₀ aryl)silyl group, a diphenylaminophenyl group, a ditolylaminophenyl group, and derivatives thereof.

17. The organic light-emitting compound of claim 13, wherein R₁, R₂, R₃, R₄, R₅, R₆, R₇, R₈, R₉, R₁₀, R₁₁, R₁₂, R₁₃, and R₁₄ are each independently selected from the group consisting of a methyl group, a cyclohexyl group, a phenyl group, a biphenyl group, a tolyl group, a naphthyl group, a pyrenyl group, a phenanthrenyl group, a fluorenyl group, an imidazolinyl group, an indolyl group, a quinolinyl group, a diphenylamino group, a N,N-diphenylaminophenyl group, a N,N-di-p-tolylaminophenyl group, a triphenylsilyl group, and derivatives thereof.

18. The organic light-emitting compound of claim 13, wherein X is CH₂, C(CH₃)₂, C(C₆H₅)₂, or C(C₆H₁₁)₂.

19. The organic light-emitting compound of claim 1, which is selected from compounds represented by Formulae 3 through 28 below:

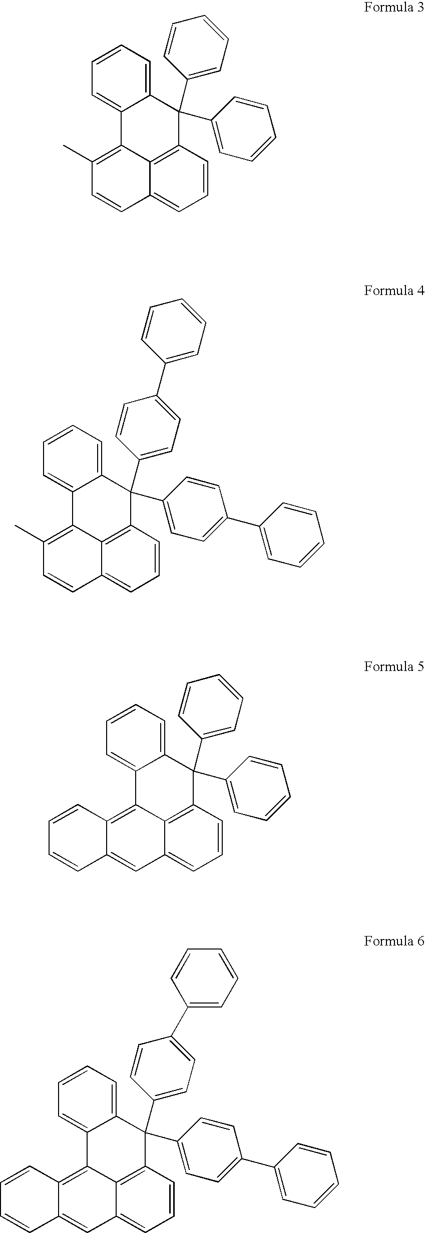

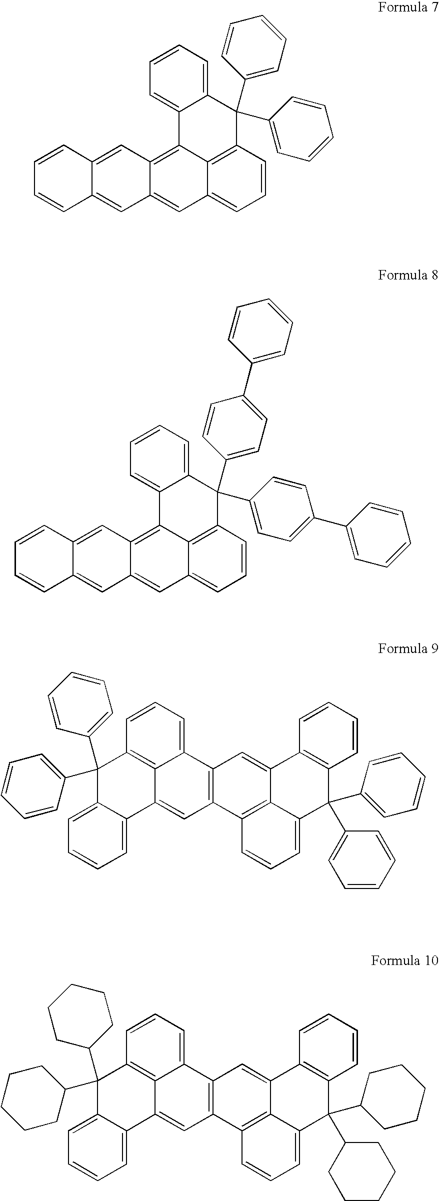

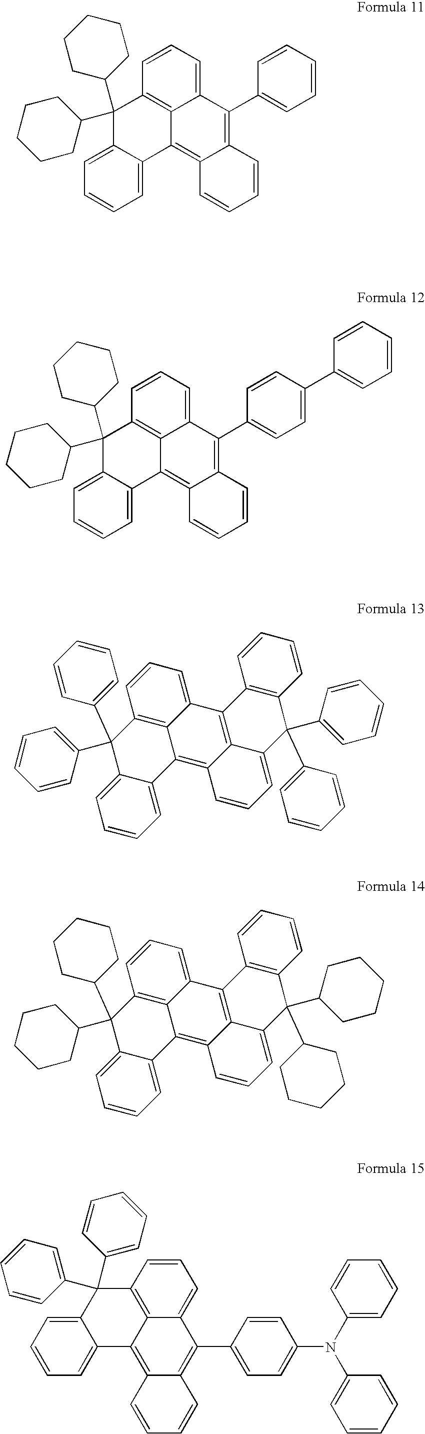

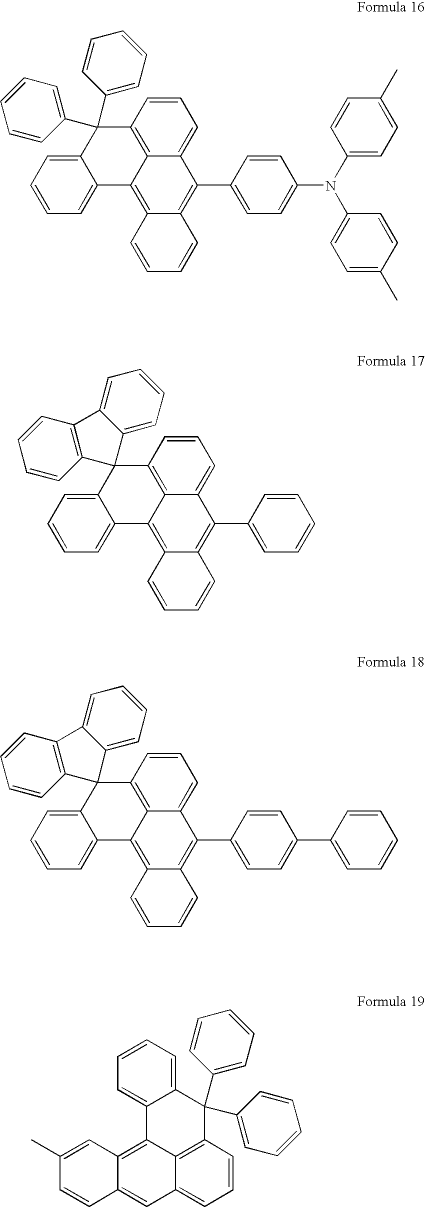

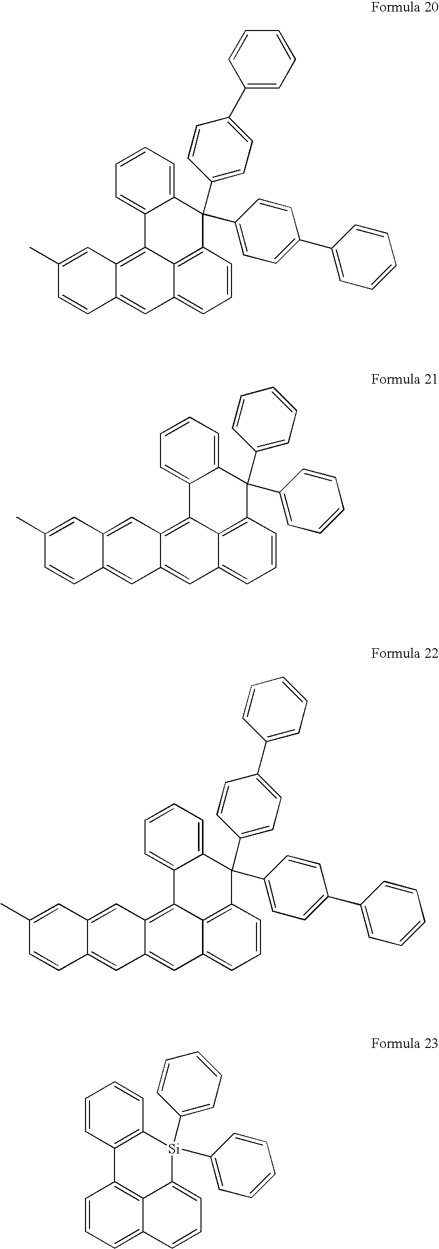

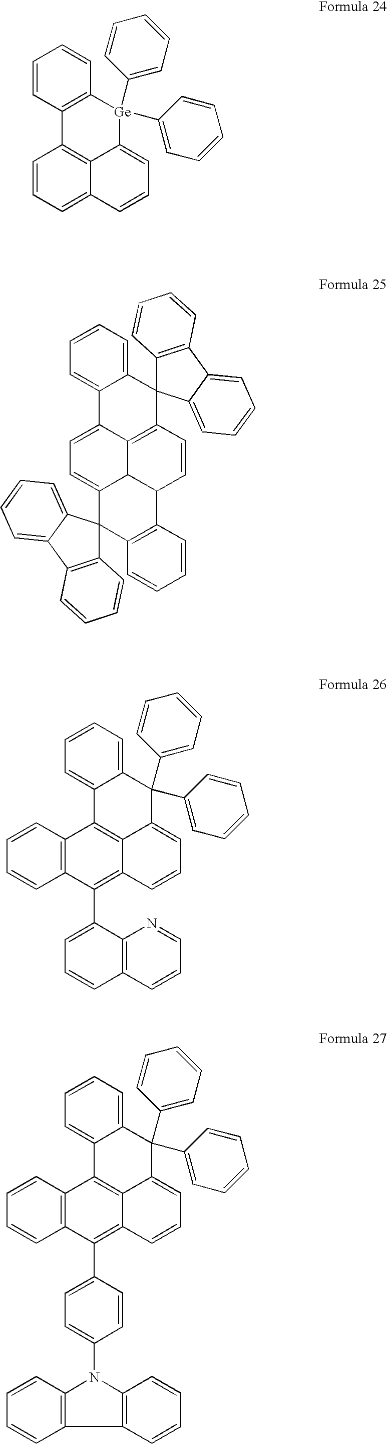

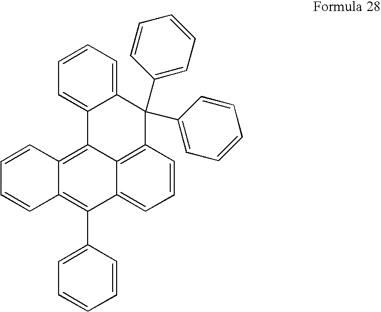

20. An organic light-emitting device comprising:
a first electrode;
a second electrode; and
at least one organic layer interposed between the first electrode and the second electrode, the organic layer comprising the compound of claim 1.

21. The organic light-emitting device of claim 20, wherein the organic layer is an emitting layer, a hole injection layer, a hole transport layer, a hole blocking layer, or an electron transport layer.

22. The organic light-emitting device of claim 20, further comprising at least one selected from the group consisting of a hole injection layer, a hole transport layer, an electron blocking layer, a hole blocking layer, an electron transport layer, and an electron injection layer, between the first electrode and the second electrode.

23. The organic light-emitting device of claim 21, further comprising at least one selected from the group consisting of a hole injection layer, a hole transport layer, an electron blocking layer, a hole blocking layer, an electron transport layer, and an electron injection layer, between the first electrode and the second electrode.

24. The organic light-emitting device of claim 22, which has a structure of first electrode/hole injection layer/emitting layer/electron transport layer/electron injection layer/second electrode, first electrode/hole injection layer/hole transport layer/emitting layer/electron transport layer/electron injection layer/second electrode, or first electrode/hole injection layer/hole transport layer/emitting layer/hole blocking layer/electron transport layer/electron injection layer/second electrode.

25. A method of manufacturing an organic light-emitting device, the method comprising:
forming a first electrode;
forming on the first electrode an organic layer comprising the compound of claim 1; and
forming a second electrode on the organic layer.

26. The method of claim 25, wherein the formation of the organic layer is performed using a dry- or wet-spray process selected from vacuum deposition, spin coating, inkjet printing, and spray printing, or a thermal transfer process.
